(19) United States
(12) Patent Application Publication
(10) Pub. No.: US 2003/0148269 A1
(43) Pub. Date: Aug. 7, 2003

(54) Centrifugally-enhanced method of determining ligand/target affinity

(76) Inventors: Thomas F. Holzman, Libertyville, IL (US); John Eric Harlan, Lake Zurich, IL (US); David A. Egan, Gurnee, IL (US); Alexander M. Buko, Winthrop Harbor, IL (US); Larry Richard Solomon, Mundelein, IL (US); Uri S. Ladror, Gurnee, IL (US); Qing Tang, Park City, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/314,861

(22) Filed: Dec. 9, 2002

Related U.S. Application Data
(63) Continuation of application No. 09/270,427, filed on Mar. 16, 1999, now abandoned.

(51) Int. Cl. C12Q 1/70; C12Q 1/68; G01N 33/53; G01N 33/567

(52) U.S. Cl. 435/5; 435/6; 435/7.2

(57) ABSTRACT

Centrifugation is used to induce and/or enhance binding between macromolecular targets and either small-molecule ligands or larger biomolecules as single entities or mixtures. With the enhanced binding, the method of the invention permits detection of ligands that bind to target substances and improve the design of ligands. The process relies on centrifugal force to establish a differential and selective concentration gradient between macromolecular therapeutic targets and the desired ligands. Once formed, the information about the self-sorting binding events is derived by analyzing the differential gradient of macromolecules and ligands in situ or by fractionating the gradient into individual samples for independent analysis. A variety of methods or combinations thereof, can be used to look for enhanced levels of bound ligands.

### TECHNICAL FIELD

The present invention relates to a method of screening organic compounds for biological activity. More particularly, the present invention concerns a centrifugally-enhanced method for screening organic compounds for their capacity and relative strength of binding to target biological molecules and for determining information about the binding regions of biologically significant compounds.

### BACKGROUND OF THE INVENTION

The discovery of new therapeutically active compounds is a long, difficult, and costly process involving the identification of therapeutically potent agents which have the requisite bioavailability, metabolic stability, and low toxicity. The first stage typically involves the screening of a library of compounds for their ability to bind to a pre-selected target biomolecule.

Classical screening methods employ assays of the ability of a test compound to bind to or otherwise inhibit an enzyme known to be involved in a metabolic pathway of interest, or to bind to a tissue binding site of interest. These assays often require significant time to develop and validate, frequently requiring information about the target biomolecule which may not be available.

In one alternative, the library of compounds to be screened can comprise a class of compounds specifically synthesized for the purpose and designed to possess the chemical structures believed to convey therapeutic activity. In an alternative approach, the library of compounds can be a group of compounds randomly synthesized, or randomly selected from a collection of compounds previously synthesized for a different purpose.

The first approach, traditionally termed “rational drug design,” involves the synthesis of a class of compounds which are modeled on a compound of known therapeutic activity. The class of compounds in the library is prepared by individually synthesizing compounds in which functional groups or structural features at certain positions in the molecule are left unchanged while a particular functional group or structural element at one specific location within the molecule is varied in a progressive manner. The compounds are individually screened in a pre-selected biological assay to determine when the functional groups or structural elements have been optimized. In this manner, a “structure activity relationship” or “SAR” matrix is constructed to find the compound having optimal therapeutic activity. This approach, while aided in recent years by the advent of “CADD” or computer assisted drug design, is still tedious and costly.

As a consequence, the narrowly focused approach of rational drug design is rapidly giving way to the second, broader approach in which a large library of compounds is randomly screened. As stated above, the screening library can comprise a large group of compounds quickly synthesized for the purpose, a library of compounds already available from another source, or a mixture of compounds from both sources. Recently developed combinatorial chemistry techniques have aided in the rapid synthesis of large libraries of compounds, and have hastened the implementation and development of the new broader approach to screening. This approach, while greatly simplifying the task of preparing the library of compounds to be screened has, on the other hand, imposed the need for quick, adaptable, and inexpensive methods for screening.

Nuclear magnetic resonance (“NMR”) spectroscopic techniques have recently been developed as an alternative to the classical assays described above as methods for determining binding of smaller molecules to macromolecular targets. U.S. Pat. Nos. 5,698,401 and 5,804,390 to Fesik, et al. describe methods in which test compounds are mixed with target proteins which have been isotopically enriched with NMR-detectable ¹⁵N nuclei. Based upon the nuclear Overhauser effect, the method permits the determination of detailed information about the site and strength of binding of the test compound to the host biomolecule. While this technique provides detailed information about the binding, it requires comparatively large quantities of isotopically enriched target macromolecule, and is generally limited to host molecules having a molecular weight less than about 30 kDa.

Another alternative to classical inhibition assays employs mass spectrometric analysis of affinity-based binding. In this method, mass spectrometry is used for the direct identification of compounds which are bound to a target biomolecule by affinity capture. Variants of this method are described by Nakanishi, et al., Biol. Mass Spectrom. 23: 230 (1994); Nelson, et al., Anal. Chem., 67: 1153 (1995); Kelly, et al., Biochem., 35: 11747 (1996); Dunayevskiy, et al., Rapid Commun. Mass Spectr., 11: 1178 (1997); and Wieboldt, et al., Anal. Chem., 69: 1683 (1997). Steinberg, et al., Biochem., 5(12): 3728 (1966) describe experiments demonstrating the interaction of bovine plasma albumin (BPA) and methyl orange by ultracentrifuge sedimentation equilibrium and sedimentation velocity methods.

### SUMMARY OF THE INVENTION

In accordance with the present invention, there is provided a method of determining affinity binding comprising the steps of first exposing at least one target substance to at least one putative ligand substance in a mixture in a container vessel. Second, the container vessel and contents are subjected to centrifugation. Third, a physical parameter is measured at each of several depths of the mixture in the container vessel which is proportional to the concentration of the at least one target substance or the at least one putative ligand substance in the mixture.

In one alternative embodiment of the method, the step of determining the physical parameter which is proportional to concentration of the at least one target substance and/or at least one putative ligand, is carried out in situ. In a second alternative embodiment, the step of determining the physical parameter is carried out by withdrawing aliquot samples for analysis from various depths of the container vessel. In yet another embodiment, the step of determining the physical parameter is carried out by separating the contents of the container vessel into individual fractions based upon depth in the container vessel subsequent to the step of centrifugation, but prior to analysis.

The target substance may be a biopolymer such as a lipid, lipoprotein, polypeptide, protein, DNA, RNA, or polysaccharide, any of which may be in either the pure or impure state. The method has applicability, inter alia, to the screening of potential therapeutic agents to determine their ability, or lack thereof, to bind to a target macromolecule of therapeutic interest. However, the method of the present invention is not limited to the detection of binding between macromolecular substances and small molecules (i.e. molecules having a molecular weight less than about I kDa), but can also be used to determine binding between and among macromolecular substances such as DNA, RNA, polysaccharides, viruses, cells and sub-cellular components including cell organelles.

For example, following the determination of binding between a virus and a cell, various components of the cell can be separated and tested for binding with the virus to determine which cell component(s) is (are) involved in the binding. In another alternative, once binding has been established between a ligand substance and a target biopolymer, the method of the present invention can be used to determine information about the specific binding region(s) in the target biopolymer.

The method of determining the physical parameter which is directly proportional to the concentration of the at least one target substance and/or at least one putative ligand substance may be by any of the techniques well known to those skilled in the art, including of mass spectrometry, nuclear magnetic resonance spectrometry, absorbance spectroscopy, fluorescence spectroscopy, atomic absorption spectroscopy, chromatography, gel electrophoresis, enzymatic assay, immunoassay, and radio-isotopic assay or combinations thereof.

The method of the present invention has a number of advantages over prior art methods of determining affinity binding. First, the method is completely independent of the structure or size of the target substance; hence there is no upper limit imposed by the method on the molecular weight of either the target substance or the putative ligand substance.

Second, the method is independent of the nature and type of target substance, so there is no need to customize target-dependent assays to determine binding affinity.

Third, the method is applicable to small amounts of both the target substance and the putative ligand substance(s), permitting in some embodiments, the detection of ligands at less than micromolar concentrations in sample volumes ranging between about 10 μL to about 100 μL.

Fourth, the method can be applied to the determination of affinity binding between putative ligand substances and the target substance in situations in which the putative ligand substance is either soluble or insoluble in the mixture containing the target substance.

Fifth, the amount of the complex formed between the target substance and ligand substance can be precisely controlled by altering the sedimentation time and rotational velocity (i.e. centrifugal field strength) during the step of subjecting the container vessel and contents to centrifugation.

### BRIEF DESCRIPTION OF THE DRAWING FIGURES

In the drawings, which form a part of this disclosure:

FIG. 1 is a plot of concentration gradients of ligand substances in a typical experiment in which binding of ligand substances to a target substance are measured by the method of the present invention.

FIG. 2A is a plot of the concentration gradients for 3-[4-cyano(1,1-biphenyl-4-yl)oxy]-N-hydroxypropanamide following centrifugation in buffer only (open circles) and in buffer in the presence of stromelysin catalytic domain (SCD), (closed squares) and illustrates the results of testing, by the method of the present invention, a ligand substance which binds strongly to a selected target substance.

FIG. 2B is a plot of the concentration gradients for 5-[4-cyano(1,1-biphenyl-4-yl)oxy]-N-hydroxypentanamide following centrifugation in buffer only (open circles) and in buffer in the presence of SCD (closed squares), and illustrates the results of testing, by the method of the present invention, a ligand substance which binds moderately to a selected target substance.

FIG. 2C is a plot of the concentration gradients for 4′-hydroxy-1,1-biphenyl-4-carbonitrile following centrifugation in buffer only (open circles) and in buffer in the presence of SCD (closed squares), and illustrates the results of testing, by the method of the present invention, a ligand substance which binds weakly to a selected target substance.

FIG. 2D is a plot of the concentration gradients for N-phenylbenzamide following centrifugation in buffer only (open circles) and in buffer in the presence of SCD (closed squares), and illustrates the results of testing, by the method of the present invention, a putative ligand substance which does not bind to a selected target substance.

FIG. 3 is a schematic representation of a typical 3-dimensional contour surface plot of the mass spectral data array derived from a spin-screen assay in accordance with one embodiment of the method of the present invention

FIGS. 4A-6B are displays of planar cuts at constant mass spectral signal intensity of three-dimensional contour plots of data arrays from mass spectrometric analysis of the type illustrated in FIG. 3. The figures are derived from experiments in which a 25-compound library was subjected to spin-screen analysis with buffer only (FIGS. 4A, 5A and 6A), and with buffer in the presence of Erm AM (FIGS. 4B, 5B and 6B).

FIGS. 4A and 4B are data displays for two soluble ligands of Erm AM.

FIGS. 5A and 5B are data displays for two insoluble ligands of Erm AM.

FIGS. 6A and 6B are data displays for the results of evaluation of binding of a 25-compound library to Erm AM. The library was made up of compounds whose capacity for binding to Erm AM was not previously known.

FIG. 7 is a display of the concentration gradient curves from a spin-screen assay of a series of GFP-EF3 fusion proteins with yeast cell ribosomes.

### DETAILED DESCRIPTION OF THE PREFERRED EMBODIMENTS

1. Terminology

As used throughout this specification and the appended claims, the following terms have the generally accepted meanings and usage.

“AEBSF” denotes 4-(2-aminoethyl)benzenesulfonyl fluoride.

The abbreviation “bp” refers to the term base pairs.

The term “Da” and “kDa” refer, respectively to the Dalton and the kiloDalton. The Dalton is one atomic mass unit.

“DNA” and “RNA” refer, respectively to deoxyribonucleic acid and ribonucleic acid.

“EF3” and “YEF3” refer, respectively to the protein, or gene for, yeast elongation factor 3.

“ERM” denotes the protein erythromycin resistant methylase.

“FKBP” refers to FK-506 binding protein.

“GFP” refers to the green fluorescent protein of Aequoria victoria.

“HPLC” refers to high performance liquid chromatography.

“MS” refers to mass spectrometry, and “ESI” and “APCI” refer, respectively to the electrospray ionization technique and the atmospheric pressure chemical ionization technique of mass spectrometry.

The terms “spin-screen” or “spin-screening” are used throughout this specification as abbreviated descriptions for the method of the invention, as that method is defined by the appended claims.

The abbreviation “nm” refers to wavelengths in nanometers, while “nM” refers to nanomolar solution concentrations, and “nt” refers to nucleotides in a DNA or RNA sequence. The abbreviation “μM” denotes micromolar solution concentrations, and “mV” refers to millivolts.

“PCR” denotes the polymerase chain reaction technique.

“PVDF” refers to polyvinylidene difluoride.

“SCD” refers to the 81-256 amino-acid internal fragment of the 447-amino acid protein, stromelysin, i.e. the stromelysin catalytic domain.

“SDS-PAGE” refers to sodium dodecyl sulfate polyacrylamide gel electrophoresis.

“Tween® 20” is the trademark of Atlas Powder Company, Ninth and Market Streets, Wilmington, Del., USA, for polyoxyethylenesorbitan monolaurate surfactant.

II. General Discussion

In accordance with the method of the present invention, centrifugation is used to induce in a mixture of target substance and putative ligands, a selective gradient of target substance and bound ligands as opposed to unbound ligands. The ligands may be comparatively small molecules (i.e. molecules having a molecular weight less than about 1 kDa), large molecules, or any mixture thereof. With the selective gradient, it is possible to detect ligands which bind to target biomolecules and to use this information to select and improve the design of such molecules. Moreover, it is possible to use the method of the present invention to gather information about the specific binding regions of a ligand and/or the target macromolecule.

The method of the present invention relies upon the establishment, by centrifugal force, of a differential and selective concentration gradient between and among the target substance and the putative ligands in the centrifuge tube container vessel. Centrifugation induces a centrifugal force outward from the center of rotation. In the centrifuge rotor, the bottom of a sample tube is furthest from the center of rotation, and the meniscus of the fluid contained in the tube is nearest the center of rotation. The centrifugal force, during rotation, thus produces a gradient of “g” force which increases incrementally from the meniscus to the bottom of the tube. Before spinning, the target substance and ligand(s) are uniformly distributed in the mixture contained in the centrifuge tube along the length of the tube from the meniscus to the bottom of the tube. In this homogenous mixture, any compounds which are capable of binding to the target substance will bind to that substance to an extent controlled by two parameters: the relative concentrations of the ligand and target substances, and the intrinsic strength of the interactions of the ligand substance with the target substance. That binding may be characterized as weak, medium, or strong. Prior to centrifugation, an equilibrium condition is achieved, and under these conditions, the ligands with the greatest intrinsic affinity for binding to the target substance produce a higher concentration of complex formed with the target substance. That is, at any instant in time, there will be a greater fractional state of strong-binding ligand to the target substance than for the medium-binding or weak-binding ligands.

When this equilibrium mixture is centrifuged for a predetermined period of time and at a predetermined rotational speed, the distribution of the macromolecular target substance achieves an exponential concentration gradient from the meniscus of the carrier fluid to the bottom of the tube. This exponential concentration gradient can define a steep curve, if a high rotational speed is employed during centrifugation, or a shallow curve in the case of low rotational speed. The concentration of target substance at and near the bottom of the centrifuge sample tube can thus be adjusted by adjusting the sedimentation time and rotational velocity.

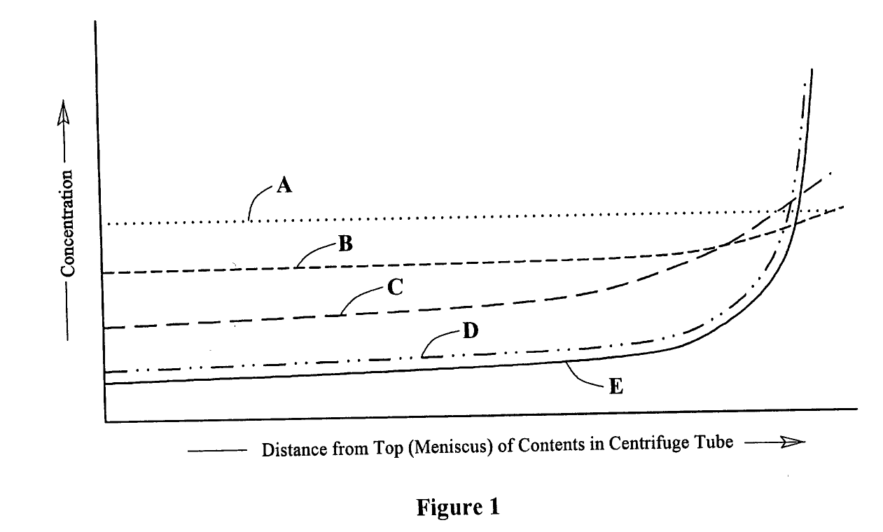

Following centrifugation, the amount of ligand substance at various depths of the centrifuge tube is measured by a suitable analytical technique. FIG. 1 depicts a typical plot of the results of a spin-screen experiment employing the method of this invention. In the figure, the vertical axis represents concentration of the ligand substance, or any physical, chemical, or biological parameter which is directly proportional to concentration. The concentration (or parameter) is measured for each ligand substance at various depths of the centrifuge tube. In the graph in FIG. 1, the horizontal axis plots the depth in the tube from which the sample as taken for analysis.

The various concentration gradient curves plotted in FIG. 1 show typical results for a substance which does not bind to the target substance (line A), one that binds weakly (line B), one that binds moderately (line C), and one that binds tightly (line D) with the target substance. The concentration gradient of the target substance following centrifugation is shown as line E.

The present invention contemplates as suitable analytical techniques for the method of the invention, the techniques of mass spectrometry, nuclear magnetic resonance spectrometry, absorbance spectroscopy, fluorescence spectroscopy, atomic absorption spectroscopy, chromatography, gel electrophoresis, enzymatic assay, immunoassay, and radio-isotopic assay. The actual concentration of the ligand substance may be, but need not necessarily be, determined. For example, in a situation involving the testing of one or a small number of ligand compounds having distinctive absorption wavelengths and whose molar extinction coefficients are known, the actual concentrations of each substance may be determined following centrifugation. If HPLC is used, the amount of each particular material in a sample can be ascertained by reference to retention times and areas under the elution curves for standard samples of the compounds.

Example 1 below illustrates this use of the method of the invention in individually testing the binding of four compounds to the 81-256 internal catalytic domain region of human stromelysin using HPLC as the analytical technique to determine the concentration gradients. Human stromelysin is a 447-amino acid protein believed to be involved in proteolytic degradation of cartilage. Cartilage proteolysis is believed to result in degradative loss of joint cartilage and the resulting impairment of joint functioning observed in both osteoarthritis and rheumatoid arthritis. The protein contains a series of domains including N-terminal latent and pro-peptide domains, a C-terminal domain homologous with homopexin, and an internal catalytic domain.

Studies have shown that removal of the N-terminal pro-sequence of eighty amino acids occurs to convert the pro-enzyme to the 45 kDa mature enzyme. Further, studies have shown that the C-terminal homopexin homologous domain is not required for proper folding of the catalytic domain or for interaction with an inhibitor. (See, e.g., A. I. Marcy, Biochem., 30: 6476 (1991). Thus, the 81-256 aminoacyl residue internal fragment (stromelysin catalytic domain, SCD, SEQ ID No. 1), of the protein was chosen as the target substance for identifying by the method of this invention compounds which bind to, and have potential as inhibitors of, stromelysin. The protein fragment was prepared by expression in a modified strain of E. coli as described below under the heading “Preparation of Starting Materials.”

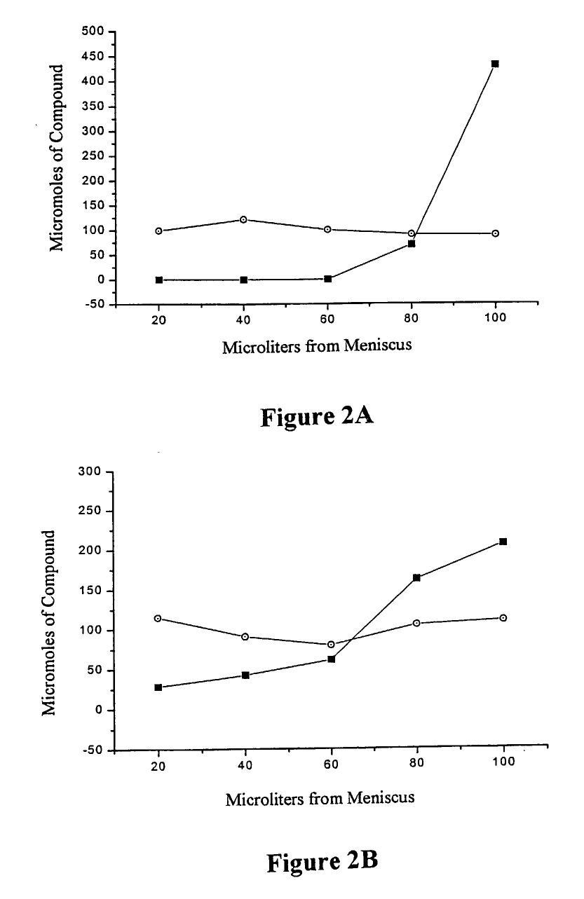

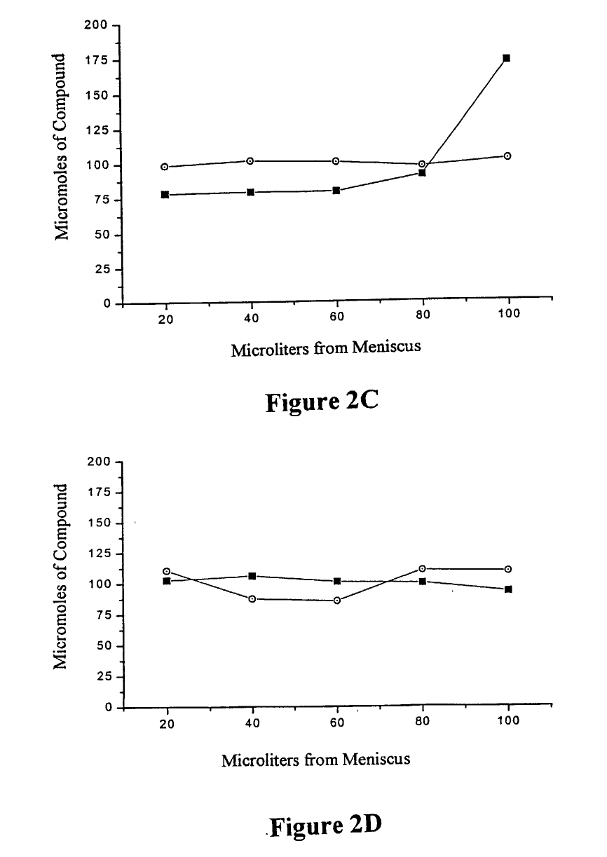

In Example 1 below, the binding affinities of four compounds were assayed for affinity binding to SCD by the method of the present invention. The compounds were chosen to illustrate the invention, since their relative affinities for SCD were known from independent measurements by another method. The compounds were subjected to spin-screen analysis by the method of the present invention using HPLC as the analytic technique for determining concentrations. The techniques employed in sample preparation, centrifugation, HPLC, and data analysis were those described under the heading “General Methodology” below. In each instance, the amount of compound at five different levels in the centrifuge tube were determined by HPLC, with comparison of the areas under the elution curves to those of standard samples. The results are shown in FIGS. 2A-2D. In each figure, the curve represented by the open circles are data from a spin-screen experiment in which the compound was spun with buffer only in the absence of SCD. The data represented by the closed squares are data from a spin-screen experiment in which the compound was spun with buffer and SCD.

The data for the compound appearing in FIG. 2A (strong binding, ˜25 nM) shows the steep upward curvature for concentrations near the bottom of the tube typical of a compound which strongly binds to SCD. The data appearing in FIG. 2B (medium binding, ˜3.5 μM) shows a general upward curvature over the length of the tube, with greater slope near the bottom of the tube. These data are typical of a compound which demonstrates medium binding affinity for SCD. FIG. 2C shows data for a compound which binds weakly (˜1 mM) to SCD, and displays the relatively flat concentration gradient over most of the tube length, with an upward slope only near the bottom of the tube typical of a weakly binding compound. Finally, FIG. 2D shows the concentration gradient data for a compound which does not bind to SCD. In FIG. 2D, the curves for the spin-screen experiments in both the presence of and the absence of SCD are flat, showing that no detectable amount of the compound was bound to and brought down with the protein by centrifugation.

Although Example 1 employed HPLC which permitted the determination of the actual amounts of compound present at each level of the centrifuges tube, generally all that is required in the method of this invention is some analytical technique which measures a physical, chemical, or biological parameter of the ligand substance which is proportional to its concentration. Such data is sufficient to construct the concentration gradients curves required for analysis of binding.

For certain analytical techniques, the ligand substance may be “tagged” prior to the experiment by a marker, such as a radio-isotopic label, a fluorescent label, or the like. On the other hand, the ligand substance may itself bear an intrinsic marker such as natural fluorescence, color, or one or more characteristic signals in its optical or nuclear magnetic resonance (NMR) spectra. A preferred method for the analytical step of the method of this invention, however, is mass spectrometry (MS). Mass spectrometry permits the direct identification of each ligand substance in a complex mixture (library) of substances subjected to spin-screen affinity analysis, and does not require that the ligand substances be tagged or possess an intrinsic tag. On the other hands, tags are advantageously employed when the substances being examined by the method of this invention are of a molecular weight too large to be easily analyzed by mass spectrometry (proteins, cells, cell fragments, cell components, DNA, RNA, etc.) Examples below illustrate the use of mass spectrometry, chromatography, and fluorescent spectrometry as analytical techniques in the method of this invention. The use of the preferred analytical method of this invention, i.e. mass spectrometry, and the treatment of the resulting data is described in detail below under the heading “General Methodology;” however, the following hypothetical example is illustrative of the technique.

For purposes of illustration, assume that the affinity binding of a mixture of compounds A, B, C, and D with a protein, P, is to be analyzed by the method of the present invention. The four putative ligands, A-D, at similar molar concentrations, are mixed with P and the mixture is centrifuged under one set of conditions of time and rotational speed (condition set “X”). The centrifuged mixture is then fractionated into five fractions comprising the one-fifth of the tube contents nearest the meniscus, the next one-fifth, etc., to the final one-fifth at the bottom of the tube. Some parameter directly proportional to the concentration of each putative ligand compound A-D in each fraction is determined, for example, by mass spectrometric analysis, HPLC, absorption or emission spectroscopy, etc.

For illustrative purposes, further assume that the results indicate nearly similar concentrations of compound A in each sample; shallow upwardly sloping concentration gradient curve for compound B; and a moderately upwardly sloping concentration gradient curve for compound C, and a steeply upwardly sloping concentration gradient curve for compound D. These results would indicate that compound A exhibits extremely weak, or non-existent, binding to protein P, compound B binds weakly to protein P, and compound C binds with moderate strength to protein P, and D binds strongly.

The method of the present invention can be easily employed in screening a library of compounds for therapeutic activity. Compound D would be selected by the researcher for further examination as a potential inhibitors of protein P, while compound A would be discarded as a potential candidate. Compounds B and C might be subjected to further analysis prior to their being discarded as candidates. While this hypothetical illustration is with a mixture of four compounds, there is no theoretical limit on the number of compounds which could be analyzed together in a single experiment. The only practical limitations on the number of putative ligands in a library sample are imposed by solubility problems and the difficulty in “sorting out” the signals for each compound in a sample containing a large library of compounds. The latter limitation varies, of course, depending upon the analytical method employed.

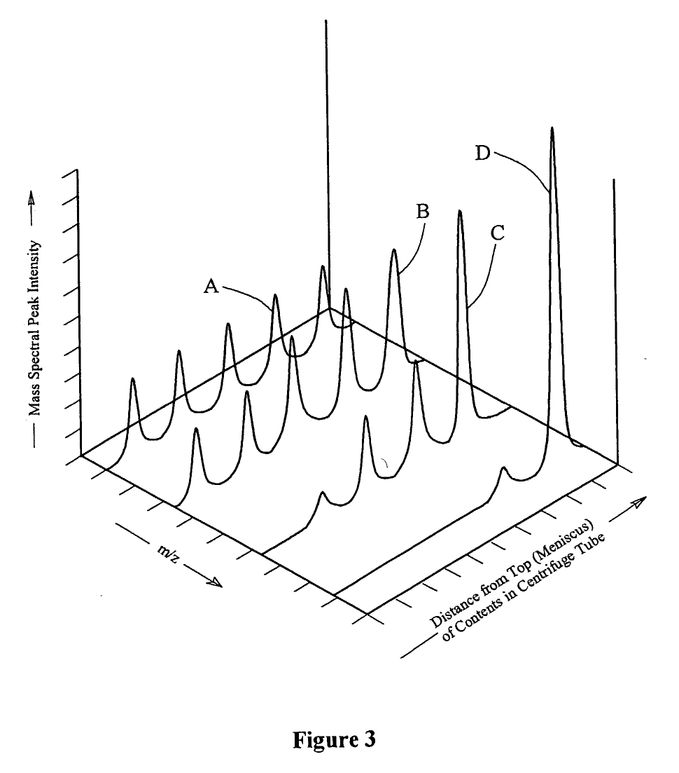

If the hypothetical experiment just described is carried out using mass spectrometry as the analytical technique, the data from such an experiment can be displayed as illustrated in FIG. 3. In that figure, the data are shown plotted as a 3-dimensional plot in which one axis is m/z, the second is distance from the meniscus of the fluid from which a sample is taken for mass spectral analysis, and the third axis is mass spectral peak intensity. (The method of data analysis is described in detail below under the heading “General Methodology.”) In FIG. 3, the tracing labeled “A” represents a compound which has not bound to the target substance. Samples withdrawn from the centrifuge tube following centrifugation shown equal or roughly equal amounts of compound A since it has not bound to, and been thrown down with, the target substance.

The tracing labeled “B” shows a shallow upward slope in the mass spectral peaks with increasing distance from the meniscus at which the sample was taken for analysis. This is typical of a compound which binds weakly to the target substance. The tracing labeled “C” is representative of a compound which binds moderately to the target substance, and tracing “D” represents a compound which binds strongly.

Assume further, for the purposes of this illustration, that the researcher wished to examine further the comparative binding efficiencies of compounds B and C. This can be done by the method of the present invention by simply repeating the experiment of this illustration under a different set of conditions of sedimentation time, rotational velocity, and initial concentration. For example, assume the experiment is repeated with compounds B and C under a set of conditions “Y” of either or both longer sedimentation time and higher rotational velocity. Since these conditions would produce a steeper gradient curve for the protein P from the fluid meniscus to the bottom of the tube, the concentration of P at the bottom of the tube would be much higher than in the experiment under condition set X. Since, as stated above, binding is dependent upon both intrinsic binding (dissociation constant) of the ligand and the protein concentration, the results of the experiment under condition set Y would produce concentration gradient curves for compounds B and C which are distinguishable. The determination could then be made whether compound B or compound C is more strongly bound to protein P.

In the case of two strongly-binding ligands, the experiment would be repeated under condition set “Z” of either or both lower sedimentation time and rotational velocity. This set of conditions would result in a more gradual concentration gradient of protein P from the meniscus of the fluid to the bottom of the tube. The compound which intrinsically binds more strongly to protein P would, under these conditions, demonstrate the more steeply sloping of the two ligand concentration gradient curves.

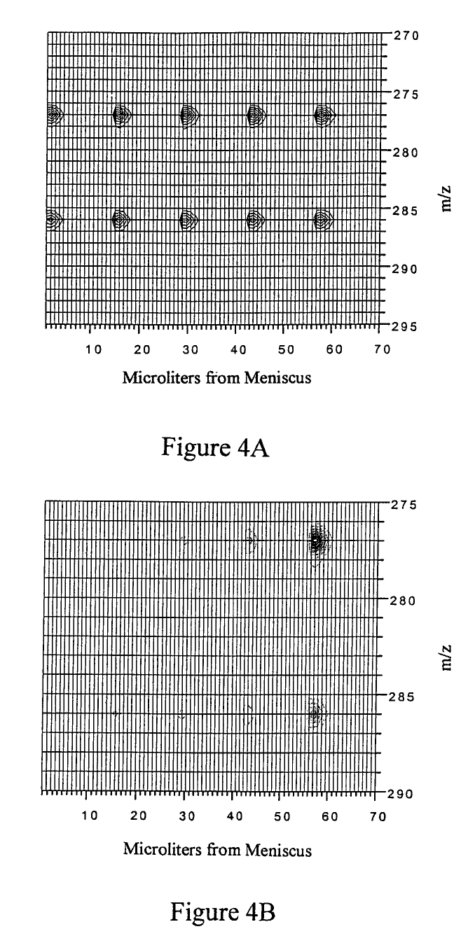

A particularly convenient manner of analyzing data from spin-screen experiments performed by the method of this invention involves viewing the data plot of FIG. 3 along the axis representing mass spectral peak intensity. A display of data for constant peak intensity results in data plots of the types shown in FIGS. 4A-6B. A contour plot of mass spectral data of the type shown in FIG. 3 can be thought of as a contour maps of chains of “mountain peaks” of varying height, where the mass spectral peaks form the “mountains” lying along a straight chain of constant m/z. The planar cuts depicted in FIGS. 4A-6B then become, in this analogy, the surface of an imaginary “sea”. The data displayed in FIGS. 4A-6B can then be thought of as the cross-sectional bases of chains of mountainous “islands” rising from the imaginary sea. In FIGS. 4A-6B, the data were taken at comparatively high values of mass spectral peak intensity, hence only “islands” corresponding to the tallest “mountains” (i.e. highest mass spectral signal intensities) are shown.

This permits the observation of data for those compounds within a multi-compound library which bind most strongly to the protein or ionize most readily and thus have the highest peak intensity. It is to be understood, however, that other compounds contained in the library which bind less strongly to the target protein, or ionize less readily, can be detected by simply lowering the water level of the imaginary “sea”, that is, by viewing the 3-dimensional plot of the data array at planar cuts at successively lower constant values of mass spectral intensity. As the value of mass spectral signal intensity is decreased for the successive planar cuts, the chains of “islands” emerge one-by-one from the imaginary “sea”; more precisely, as the data are displayed at progressively lower values of mass spectral peak intensity, the planar-cut data display reveals additional ligands at each successively lower value of m/z. In this analogy, compounds which bind strongly to the target substance would be represented by “island chains” with the taller “mountains” from samples taken near the bottom of the centrifuge tube and no “mountain” observed at the top of the tube. Compounds which do not bind would be represented by “island chains” in which the mountains on the “island” chain are of roughly equal height. Compounds of medium or weak binding would be represented as “island chains” intermediate between these extremes.

Examples 2(1) and 2(2) illustrate the use of this invention to detect affinity binding in situations where the ligands which bind to the target substance are soluble (Example 2(1)) and insoluble (Example 2(2)) in the carrier solvent system employed in the centrifugation step. The data from these experiments is presented in FIGS. 3A and 3B.

In Example 2(1), a library of 25 compounds was tested for affinity binding to erythromycin resistant methylase (Erm-AM). Two compounds, namely N-cyclohexyl-6-( 1-piperidinyl)-1,3,5-triazine-2,4-diamine (m/z=277) and (4-[4-amino-6-(cyclohexylamino)-1,3,5-triazin-2-yl]phenol (m/z=286), both soluble in the carrier solvent, were found to strongly bind to Erm-AM. FIG. 4A shows the results of data analysis from the spin-screen experiment with the 25-compound library spun in the absence of the protein, and FIG. 4B in the presence of the protein.

In FIG. 4, data plots are shown for a spin-screen experiment in which the two compounds, which were soluble in the buffer system employed, were detected to bind to Erm AM. Centrifugation of the sample mixture was carried out under conditions of sedimentation time and rotational velocity insufficient to clear the solution of the low molecular weight compounds in the sample library. FIG. 4A shows the uniform distribution of the compounds throughout the centrifuge tube. The scan numbers represent sequential scans taken in the mass spectrometer as each of the five equal fractions were analyzed. The “islands,” representing the intensities for a given value of m/z in a given fraction, taken sequentially from the top of the tube (˜scan 1) to the bottom of the tube (˜scan 57), are essentially equal in size.

FIG. 4B shows, on the other hand, the data from the spin-screen experiment in the presence of Erm AM. The data plot shows no “island” from the sample taken at the top of the tube (˜scan 1) for either compound, but shows “islands” which grow in area for both compounds in the progression from the second fraction (˜scan 15) to the bottom of the tube (˜scan 57). These data show that the two compounds were both bound to, and carried down with, the protein during centrifugation. The data also clearly show that the compound at m/z=277 bound more strongly to Erm-AM than the compound at m/z=286.

In Example 2(2), a library of 25 compounds was similarly tested for affinity binding to erythromycin resistant methylase (Erm-AM). Two compounds, namely N′cyclohexyl-N-[3-(2,4-dichloro-6-hydroxyphenyl)propyl]-N-methyl-1,3,5triazine-2,4,6-triamine and N-[3-(2,4-dichloro-6-hydroxyphenyl)propyl]-N′-(2,3-dihydro-1H-inden-2-yl), both insoluble in the carrier solvent were found to strongly bind to Erm-AM.

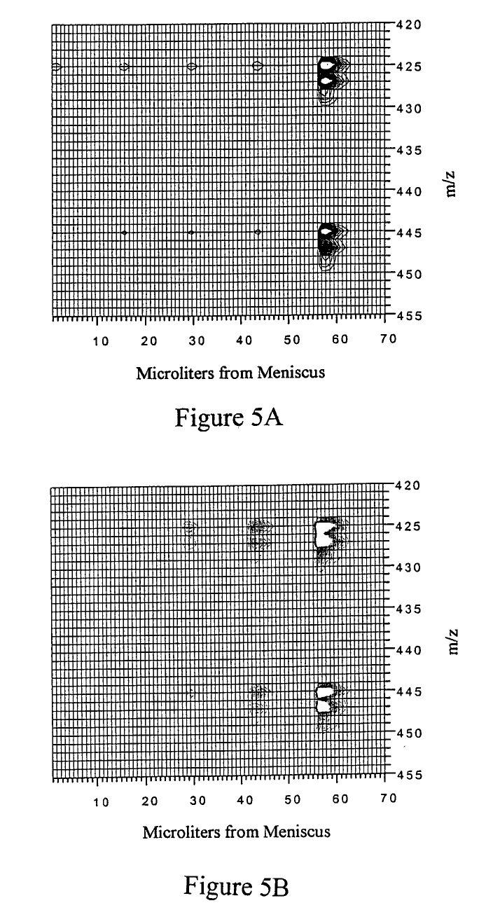

FIG. 5 show concentration gradients for the two, with FIG. 5A representing the spin-screen experiment in the absence of protein, and FIG. 5B in the presence of the protein. The signal doubling in the mass spectra of the compounds was due to the presence of chlorine atoms in each molecule and the approximately equal isotopic abundance of ³⁵Cl and ³⁷Cl. For the experiment without protein (FIG. 5a), the data plot shows that most of the compounds are deposited in the bottom-most fraction of the centrifuge tube, as would be expected following centrifugation for compounds insoluble in the carrier solvent used in the experiment. However, in FIG. 5B, where the compounds were spun with protein, the plots exhibit the more exponential concentration gradient typical of a compound which binds to the protein. In this display, the compound appears to be deleted from the uppermost layer of the centrifuge tube, but the signal “islands” from fractions 3 and 4 of the sample tube are enriched with compound when compared with the corresponding signals in FIG. 5A. In this case, binding of the compounds with the protein have solubilized them, thus retaining material at higher elevations in the centrifuge tube after centrifugation. The data displayed in FIG. 5B thus confirm that the two compounds bind to Erm.

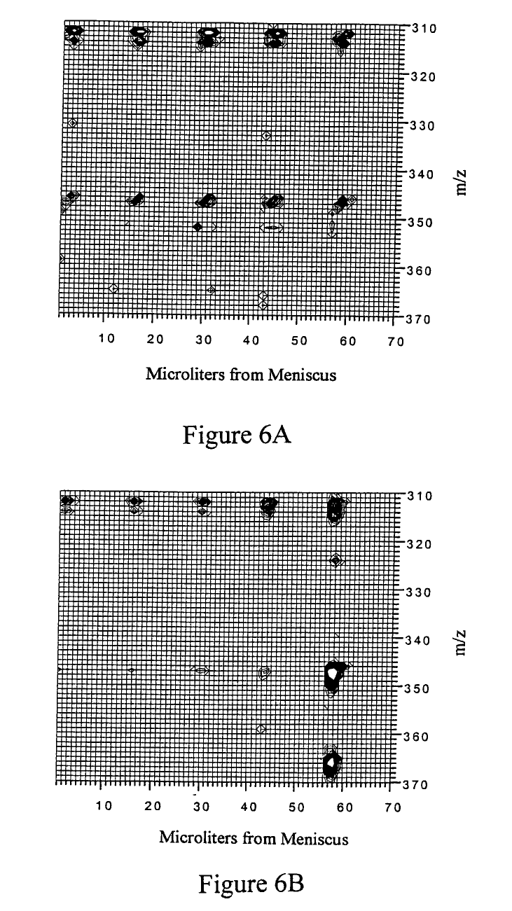

Example 3 illustrates the use of the method of the present invention to screen a 25-compound library for the ability of each compound to bind to Erm AM. The binding properties of all compounds in the library for Erm AM were unknown prior to the commencement of the experiment. FIGS. 6A and 6B show the results of spin-screen experiment in the presence of, and the absence of the protein, respectively.

Examination of the data appearing in FIG. 6A shows that the compound at m/z=312, 314, namely 4-amino-6-chloro-1,3benzenesulfonamide, is soluble in the buffer, and demonstrates the characteristic “flat” concentration gradient in the centrifuge tube in the absence of the protein. In FIG. 6B, however, there is a slight increase in concentration of the compound in the bottom of the tube, indicating medium binding to the protein.

The data “islands” for the compound, 4-[[[[3-(trifluoromethyl)phenyl]amino]-carbonyl]amino]benzoic acid appearing at m/z=324 and 1,2-benzenedicarboxylic acid, mono[(3-chlorophenyl)phenylmethyl] ester, at m/z=366 in FIG. 6B (i.e. the spin-screen experiment in the presence of protein) are essentially absent from the data plot in FIG. 6A (i.e. the spin-screen experiment in the absence of protein). While not holding to one theory to the exclusion of others, it is believed that these two compounds, in the absence of protein, may have bound to the centrifuge tube walls, pipette walls, or other surfaces and were lost in this, or some other manner, prior to mass spectral analysis. In the experiment when the protein was present, however (FIG. 6B), a portion of each compound bound to the protein and subsequently appeared in the mass spectral data. In any event, the data appearing in FIG. 6B show that the compound at m/z=324 bound weakly to the protein, while that at m/z=366 bound strongly. Finally, the compound at m/z=346, 4-hydroxy-1-[4-(phenylmethoxy)phenyl]-2(1H)-quinoline, represents a compound soluble in the carrier solvent and strongly bound to Erm AM as can be seen by the data appearing in FIGS. 6A and 6B. Binding was subsequently confirmed by independent means.

Example 4 illustrates two further applications of the method of the present invention. First, the example illustrates the use of the method to analyze affinity binding between a macromolecular ligand substance and a macromolecular target substance. Second, the method illustrates how the method the present invention can be used to elicit information about a region of a protein required for binding to a pre-determined target substance. In the experiment, the binding of yeast elongation factor 3 (EF3) to yeast cell ribosomes was studies by the spin-screen method of the present invention, employing fluorescence spectroscopy as the method of analyzing the amount of ligand protein in each fraction sample from the centrifuge tube.

Fungal protein synthesis is unique in requiring, in addition to EF1 a and EF2, a third elongation factor, EF3. The 116 kDa product of the YEF3 gene, EF3 has been shown to be essential for growth of yeast. This unique and essential requirement of EF3 make it an attractive target for antifungal agents. It is believed that the functioning of EF3 involves its specific interaction with ribosomes. Factors which inhibit this activity result in defective translation at the ribosome. The region of EF3 involved in ribosome binding is not completely understood, however, several lines of evidence suggest that the highly charged C-terminal region of the protein is involved.

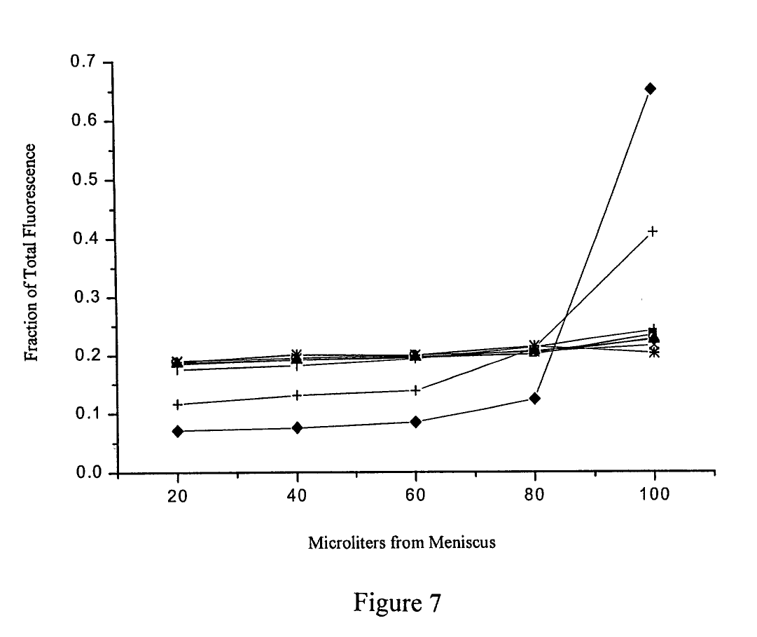

In Example 4, several C-terminus fragments of EF3 from Saccharomyces cerevisiae, fused with the green fluorescent protein (GFP) of Aequoria victoria, were expressed in modified E. coli and individually tested for binding to ribosomes. In one experiment, the fusion proteins were centrifuged in the absence of ribosomes and found to be uniformly distributed in the centrifuge tube following centrifugation. Each fusion protein fragment was then subjected to spin-screen analysis, together with yeast ribosomes, and the concentration gradient of the fusion protein fragments analyzed by fluorescence spectroscopy following centrifugation. The green fluorescent protein C-terminal EF3 fusion protein fragments are shown in Table 1 (using the standard single-letter designation for each aminoacyl residue), and the results of the spin-screen experiments appear in FIG. 7.

TABLE 1

C-Terminal EF3 Fusion Protein
Fragments Tested for Binding to Ribosomes

| | |
|---|---|
| Fragment | Sequence |

| | | |
|---|---|---|
| GFP-980- | SGQGAGPRIEKKEDEEDKFDAMGNKIAGGKKKKKLSSAELRKK | (SEQ ID No. 16) |
| 1044 | KKERMKKKKELGDAYVS SDEEF | |
| GFP-980- | SGQGAGPRIE KKEDEEDKFDAMGNKIAGGK | (SEQ ID No. 29) |
| 1031 | KKKKLSSAELRKKKKERMKKKK | |
| GFP-980- | SGQGAGPRIE | (SEQ ID No. 31) |
| 1025 | KKEDEEDKFDAMGNKIAGGKKKKKLSSAELRKKKKE | |
| GFP-980- | SGQGAGPRIEKKEDEEDKFDAMGNKIAGGKKKKKLSSAEL | (SEQ ID No. 33) |
| 1019 | | |
| GFP-980- | SGQGAGPRIE KKEDEEDKEDAMGNKIAGGKKKKK | (SEQ ID No. 35) |
| 1013 | | |
| GFP-980- | SGQGAGPRIE KKEDEEDKFDAMGNKIAGG | (SEQ ID No. 37) |
| 1008 | | |
| GFP-980- | SGQGAGPRIE KKEDEEDKFDAMGNKIAGGERMKKKK | (SEQ ID No. 39) |
| 1008:1025- | | |
| 1031 | | |

Referring to the data from the spin-screening experiments depicted in FIG. 7, the concentration gradients for protein fragments GFP-980-1044 (“+” signs) and GFP-980-1031 (closed diamonds) show the upwardly-curving concentration gradient curves characteristic of binding to the ribosomes. The concentration gradient plots of all of the shorter fragments are essentially flat and superimposed at 0.2 fraction of total fluorescence, indicating a lack of binding to the ribosomes. These data indicate that the Gln-Arg-Met-Lys-Lys-Lys-Lys (ERMKKKK) sequence at residues 1025-1031 in EF3 is required for binding to the ribosome.

III General Methodology

A. Sample Preparation

Proteins and protein fragments were isolated from recombinant material produced in E. coli as described in detail below. Proteins and protein fragments were dialyzed into standard buffers comprising either a) a mixture of 20 mM ammonium bicarbonate and 1 mM sodium azide, the pH adjusted to pH 7.2 by the addition of acetic acid, or b) 50 mM Tris-HCl, 30 mM KCl, 10 mM magnesium acetate, 10 mM 2-mercaptoethanol, 1 mM sodium azide, and 10% glycerol, pH adjusted to 7.8. In the case of the first buffer system, additional ingredients, specific to each protein, were added to the buffer mixture which included 5 mM 2-mercaptoethanol for erythromycin resistant methylase (Erm-AM), and both 5 mM 2-mercaptoethanol and 10 mM calcium chloride for the stromelysin catalytic domain (SCD). Initial protein or protein fragment concentrations ranged between 100 μM and 1 mM. These buffer mixtures were found to be compatible with direct analysis by mass spectrometry.

Test compounds were made up in concentrated solutions in dimethyl sulfoxide and diluted into the protein or protein fragment buffer solutions such that the final concentration of dimethyl sulfoxide in the buffer solutions was 1% volume/volume. As a result, each compound in the mixture of test compounds had equal access to available binding sites on the target molecule.

B. Centrifugation

The compounds were added to the protein or buffer solutions in 7 mm×20 mm polycarbonate centrifuge tubes, mixed by vortexing, and placed in the centrifuge. Each mixture was then subjected to centrifugation at a speed and for a period of time sufficient to clear the solution of macromolecular target, but not of ligand. Centrifugation was carried out either in a Model TL-100 ultracentrifuge at 100,000 rpm using a TLA-100 rotor, or in a Model L-70 ultracentrifuge at 42,000 rpm using a type 42.2 rotor. (Both centrifuges are manufactured by Beckman Instruments, 1050 Page Mill Road, Palo Alto, Calif. 84304, USA.)

Clearing time for each protein was calculated from the sedimentation coefficient, S_{20,w}, of the protein molecule and the clearing factor, k, of the rotor. The sedimentation factors were estimated from standard proteins of known molecular weight after Lemaire, et al., Anal. Biochem., 106: 12 (1980) and Lemaire, et al., Anal. Biochem., 154: 525 (1986). The clearing factor, k is estimated by Equation (1):

k=[((1n(Tₘₐₓ/Rₘᵢₙ))/ω²][10¹³/3600] Eqn. (1)

where ω is the angular velocity of the rotor in radians per second (i.e. 0.105×rpm), Rₘₐₓ is the distance from the center of the rotor to the bottom of the sample tube, and Rₘᵢₙ is the distance from the center of the center of the rotor to the meniscus of the sample solution. The clearing time, t, in hours was calculated by Equation (2):

t=k/S_{20,w} Eqn. (2)

Following centrifugation, the tubes were removed from the rotor and the tube contents divided into equal volume fractions. Any reasonable number of fractions may be employed; typically four or five fractions are used. To take the fractions, a pipette was carefully inserted into the liquid column in the sample tube to a point just below the liquid meniscus. As liquid was removed from the tube, the mouth of the pipette was slowly lowered to keep it just below the liquid surface to avoid drawing air into the pipette. The fractions were individually dispensed into microfuge tubes for analysis.

C. Fraction Analysis

### 1. By High Performance Liquid Chromatography (HPLC)

Samples for HPLC analysis were diluted 1:5 with 0.1% aqueous trifluoroacetic acid solution. Reverse-phase HPLC was performed on 80% of each diluted sample using a 3.9 mm×300 mm Waters μBondpak™ C₁₈ column (Waters Corporation, 34 Maple Street, Milford, Mass. 01757, USA) in one or the other of two systems. In System 1, two Beckman Model 110B solvent pumps, a Beckman Model 166 variable wavelength detector, and a Beckman Model 507 auto-sampler were controlled by a Beckman Model 406 analog interface. In System 2, a Beckman Model 125 programmable solvent module, a Beckman Model 168 diode array detector, and a Beckman 507 auto-sampler were controlled by Beckman Model 406 analog interface.

Elution of the compounds from the column was detected by monitoring absorbance of the eluate at either 215 nm (System 1) or at 215 nm and 280 nm (System 2). Elution times and amount of compound per unit area of the elution curves were determined for both the test compound and the proteins and protein fragments using standard solutions. Peak areas were used to determine the amount of compound and protein in each of the aliquot fractions from each tube.

### 2. By Mass Spectrometry

Mass spectrometric analysis of the samples was performed using either electrospray ionization (ESI) or atmospheric pressure chemical ionization (APCI) techniques. The ESI technique employed either a Model TSQ-700 or a Model LCQ mass spectrometer, and the APCI technique employed a Model LCQ mass spectrometer (both instruments manufactured by the Finnigan Corporation, 355 River Oaks Parkway, San Jose, Calif. 95134, USA).

For ESI analysis, fraction samples were diluted 1:5 with 50% aqueous acetonitrile, leaving the protein or protein fragment in solution. Ten-microliter aliquots, each representing about 10% of the sample, were introduced individually into the instrument by flow injection using 50% aqueous acetonitrile as the carrier. For initial studies with APCI, fractions were likewise diluted 1:5 with 50% aqueous acetonitrile. However, it was found that there was a high background in samples collected near the bottom of the tube when analyzed by the APCI technique using this diluent. A similar high background was not observed in samples analyzed by the ESI technique. While not holding to one theory to the exclusion of others, it was believed that this effect was due to protein fragmentation during ionization in the spectrometer in the APCI technique. To reduce this background, subsequent samples analyzed by the APCI technique were diluted 1:5 with neat acetonitrile which caused the proteins or protein fragments to release their bound test compounds and precipitate. Sample solutions were then clarified by centrifugation in a microfuge for five minutes prior to analysis by APCI. With the APCI technique, signals in both the positive and negative ion modes for most of the compounds tested were increased substantially by replacement of the 50% aqueous acetonitrile mass spectrometer carrier solvent with a mixture consisting of 70% methanol and 30% 0.1M aqueous ammonium hydroxide.

### 3. By Fluorescence Spectroscopy

To demonstrate the use of fluorescence spectroscopy for detection of compounds in the centrifugally-enhanced affinity analysis method of the present invention, several amino-terminus truncated fragments of yeast elongation factor 3 (EF3) were expressed in E. coli as fusion proteins with the green fluorescent protein (GFP) of Aequoria victoria using methods detailed below.

Four fractions, each comprising one-fifth of the sample, were removed from each centrifuge tube sample by means of a pipette in the manner described above and dispensed into individual microfuge tubes. The centrifuge tube was washed out with a volume of buffer equal in volume to that remaining in the centrifuge tube to form the fifth fraction. Ten-microliter aliquots of the first four factions were individually diluted to 300 μL and a 20 μL aliquot of the fifth fraction was diluted to 300 μL prior to spectrofluorometric analysis.

The fluorescence of the GFP-EF3 fusion protein in each of the five fractions was examined using an SLM-Abt-2 spectrofluorometer (SLM-Aminco, Champaign, Ill., USA). In earlier experiments, emission was monitored at wavelengths ranging from 500 nm to 570 nm, with excitation at 488 nm. The slit width was 4 nm for both excitation and emission. In later experiments, emission at 512 nm with excitation at 488 nm was observed, again using a 4 nm slit width for both excitation and emission.

D. Data Analysis

Analysis of data for the concentrations of protein or protein fragments and compounds from each centrifuge tube fraction were straightforward, when analyzed for multiple-compound/target-molecule interactions by HPLC, or when analyzed for single-compound/target-molecule interactions by mass spectrometry. However, analysis of data sets was more problematic from experiments with samples containing multiple compounds monitored full scale (i.e. 100-750 m/z) by mass spectrometric methods. Since about 140 full scale mass spectrometric scans were taken of each of the five centrifuge tube fractions, the data set for each experiment comprised some 500,000 data files. Examination of the individual intensity profiles at each value of m/z presented a daunting task. To solve this problem, a simple computer program was written to sort the text file output from the LCQ mass spectrometer by molecular weight, and to average the data over a number of scans, placing the results in one array. A three-dimensional surface contour plot of the resulting data was then made using the Origin™ software program (Microcal Software, Inc., One Roundhouse Plaza, Northampton, Mass. 01060, USA). In the plot, the X-axis represented molecular weight, the Y-axis represented scan number (corresponding to fraction number or position of the fraction in the centrifuge tube), and the Z-axis represented mass spectrometric signal intensity. A representative example of such a three-dimensional surface plot appears in FIG. 1.

III. Preparation of Starting Materials

A. Preparation of Stromelysin Catalytic Domain (SCD)

Human stromelysin is a 447-amino acid protein believed to be involved in proteolytic degradation of cartilage. Cartilage proteolysis is believed to result in degradative loss of joint cartilage and the resulting impairment of joint function observed in both osteoarthritis and rheumatoid arthritis. The protein possesses a series of domains including N-terminal latent and propeptide domains, a C-terminal domain homologous with homopexin, and an internal catalytic domain.

Studies have shown that removal of the N-terminal prosequence of approximately eighty amino acids occurs to convert the proenzyme to the 45 kDa mature enzyme. Furthermore, studies have shown that the C-terminal homopexin homologous domain is not required for proper folding of the catalytic domain or for interaction with an inhibitor. (See, e.g., A. I. Marcy, Biochemistry, 30: 6476 (1991)). Thus, the 81-256 amino acid residue internal segment of stromelysin was selected as the protein fragment for use in identifying compounds by the method of the present invention which bind to and have the potential as acting as inhibitors of stromelysin.

The 81-256 fragment (SEQ ID NO: 1) of stromelysin (SCD) was prepared by inserting a plasmid which coded for the production of the protein fragment into an E. coli strain and growing the genetically-modified bacterial strain in a suitable culture medium. The protein fragment was isolated from the culture medium, purified, and subsequently used in the centrifugally-enhanced analysis of its affinity with test compounds in accordance with the method of this invention. The procedures for the preparation processes are described below.

Human skin fibroblasts (ATCC No. CRL 1507) were grown and induced using the procedure described by Clark et al., Archiv. Biochem. and Biophys. 241: 36 (1985). Total RNA was isolated from 1 g of cells using a RNAgents® Total RNA Isolation System Kit (Promega Corp., 2800 Woods Hollow Road, Madison, Wis. 53711, USA) following the manufacturer's instructions. A 1 μg portion of the RNA was heat denatured at 80° C. for five minutes and then subjected to reverse transcriptase PCR using a GenAmp® RNA PCR kit (Applied Biosystems/Perkin-Elmer, 761 Main Avenue, Norwalk, Conn. 06859, USA) following the manufacturer's instructions.

Nested PCR was performed using first primers (a) GAAATGAAGAGTCTTCAA (SEQ ID NO: 2) and (b) GCGTCCCAGGTTCTGGAG (SEQ ID No. 3) and thirty-five cycles of 94° C., two minutes; 45° C., two minutes; and 72° C., three minutes. This was followed by reamplification with internal primers (c) TACCATGGCCTATCCATTGGATGGAGC (SEQ ID NO: 4) and (d) ATAGGATCCTTAGGTCTCAGGGGA GTCAGG (SEQ ID NO: 5) using thirty cycles under the same conditions described immediately above to generate a DNA sequence coding for amino acid residues 1-256 of human stromelysin.

The PCR fragment was then cloned into PCR cloning vector pT7BIue® (Novagen, Inc., 597 Science Drive, Madison, Wis., USA 53711) according to the manufacturer's instructions. The resulting plasmid was cut with Ncol and BamHI and the stromelysin fragment was subcloned into the expression vector pET3d (Novagen), again using the manufacturer's instructions.

A mature stromelysin expression construct coding for amino acid residues 81-256 plus an initiating methionine aminoacyl residue was generated from the 1-256 expression construct by PCR amplification. The resulting PCR fragment was first cloned into the pT7BIue® vector (Novagen) and then subcloned into the pET3d vector (Novagen), using the manufacturer's instructions in the manner described above, to produce plasmid (pETST-81-256). This final plasmid is identical to that described by Qi-Zhuang et al., Biochemistry, 31: 11231 (1992) with the exception that the present codes for a peptide sequence beginning two amino acids earlier, at position 81 in the sequence of human stromelysin. Plasmid pETST-81-256 was transformed into E. coli strain BL21(DE3)/pLysS (Novagen) in accordance with the manufacturer's instructions to generate an expression strain, BL21(DE3)/pLysS/pETST-255-1.

A preculture medium was prepared by dissolving 1.698 g of NaH₂PO₄.7H₂O, 0.45 g of KH₂PO₄, 0.075 g NaCl, 0.150 g 15 NH₄Cl, 0.300 glucose, 300 μL of 1M aqueous MgSO₄ solution and 15 μL of aqueous CaCl₂ solution in 150 mL of deionized water. The resulting solution of preculture medium was sterilized and transferred to a sterile 500 mL baffle flask. Immediately prior to inoculation of the preculture medium with the bacterial strain, 150 μL of a solution containing 34 mg/ml, of chloramphenicol in 100% ethanol and 1.5 mL of a solution containing 20 mg/mL of ampicillin were added to the flask contents. The flask contents were then inoculated with 1 mL of glycerol stock of genetically modified E. Coli, strain BL21(DE3)/pLysS/pETST-255-1. The flask contents were shaken (225 rpm) at 37° C. until an optical density of 0.65 was observed.

A fermentation nutrient medium was prepared by dissolving 113.28 g of Na₂HPO₄.7112O, 30 g of KH₂PO₄, 5 g NaCl and 10 mL of 1% DF-60 antifoam agent in 9604 mL of deionized water. This solution was placed in a New Brunswick Scientific Micros Fermentor (Edison, N.J.) and sterilized at 121° C. for 40 minutes. Immediately prior to inoculation of the fermentation medium, the following pre-sterilized components were added to the fermentation vessel contents: 100 mL of a 10% aqueous solution of NH₄Cl, 100 mL of a 10% aqueous solution of glucose, 20 mL of an aqueous 1M solution of MgSO₄, 1 mL of an aqueous 1M CaCl₂ solution, 5 mL of an aqueous solution of thiamin hydrochloride (10 mg/mL), 10 mL of a solution containing 34 mg/mL of chloramphenicol in 100% ethanol and 1.9 g of ampicillin dissolved in the chloramphenicol solution. The pH of the resulting solution was adjusted to pH 7.00 by the addition of an aqueous solution of 4N H₂SO₄.

The preculture of E. coli, strain BL21(DE3)/pLysS/pETST-255-1, from the shake flask scale procedure described above was added to the fermentor contents and cell growth was allowed to proceed until an optical density of 0.48 was achieved. During this process, the fermentor contents were automatically maintained at pH 7.0 by the addition of 4N H₂SO₄ or 4N KOH as needed. The dissolved oxygen content of the fermentor contents was maintained above 55% air saturation through a cascaded loop which increased agitation speed when the dissolved oxygen content dropped below 55%. Air was fed to the fermentor contents at 7 standard liters per minute (SLPM) and the culture temperature was maintained at 37° C. throughout the process.

The cells were harvested by centrifugation at 17,000×g for 10 minutes at 4° C. and the resulting cell pellets were collected and stored at −85° C. The wet cell yield was 3.5 g/L. Analysis of the soluble and insoluble fractions of cell lysates by sodium dodecyl sulfate polyacrylamide gel electrophoresis (SDS-PAGE) revealed that approximately 50% of the stromelysin was found in the soluble phase.

The stromelysin fragment prepared as described above was purified employing a modification of the technique described by Ye, et al., Biochemistry, 31: 11231 (1992). The harvested cells were suspended in 20 mM Tris-HCl buffer (pH 8.0), sodium azide solution containing 1 mM MgCl₂, 0.5 mM ZnCl₂, 25 units/mL of Benzonase® enzyme (Benzon Pharma A/S, Langebjerg 1, DK-4000, Roskilde, Denmark), and an inhibitor mixture made up of 4-(2-aminoethyl)benzenesulfonyl fluoride (“AEBSF”) Leupeptin®, Aprotining and Pepstatin® (all at concentrations of 1 μg/mL. AEBSF, Leupeptin®, Aprotinin®, and Pepstatin® are available from American International Chemical, 17 Strathmore Road, Natick, Mass. 01760.) The resulting mixture was gently stirred for one hour and then cooled to 4° C. The cells were then sonically disrupted using a 50% duty cycle. The resulting lysate was centrifuged at 14,000 rpm for 30 minutes and the pellet of insoluble fraction frozen at −80° C. for subsequent processing (see below).

Solid ammonium sulfate was added to the supernatant to the point of 20% of saturation and the resulting solution loaded onto a 700 mL phenyl sepharose fast flow (phenyl-Sepharose FF) column (Pharmacia Biotech., 800 Centennial Ave., P. O. Box 1327, Piscataway, N.J., USA 08855). Prior to loading, the sepharose column was equilibrated with 50 mM Tris-HCl buffer (pH 7.6 at 4° C.), 5 mM CaCl₂, and 1M (NH₄)₂SO₄. The loaded column was eluted with a linear gradient of decreasing concentrations of aqueous (NH₄)₂SO₄ (from 1M down to 0M) and increasing concentrations of aqueous CaCl₂ (from 5 mM to 20 mM) in Tris-HCl buffer at pH 7.6. The active fractions of eluate were collected and concentrated in an Amicon stirred cell (Amicon Inc., 72 Cherry Hill Drive, Beverly, Mass. 01915). The concentrated sample was dialyzed overnight in the starting buffer used with phenyl-Sepharose FF column, 50 mM Tris-HCl (pH 8.2 at 4° C.) with 10 mM CaCl₂.

The dialyzed sample was then loaded on the Q-Sepharose FF column and eluted with a linear gradient comprising the starting buffer and 200 nM NaCl. The purified soluble fraction of the stromelysin fragment was concentrated and stored at 4° C.

The pellet, representing the insoluble material from the harvested cells, was solubilized in 8M guanidine-HCl. The solution was centrifuged for 20 minutes at 20,000 rpm and the supernatant was added dropwise to a folding buffer comprising 50 mM Tris-HCl (pH 7.6), 10 mM CaCl₂, 0.5 mM ZnCl₂ and the inhibitor cocktail of AEBSF, Leupeptin(R) Aprotinin(R) and Pepstatin(R) (all at concentrations of 1 μg/mL. The volume of folding buffer was ten times that of the supernatant. The mixture of supernatant and folding buffer was centrifuged at 20,000 rpm for 30 minutes. The supernatant from this centrifugation was stored at 4° C. and the pellet was subjected twice to the steps described above of solubilization in guanidine-HCl, refolding in buffer, and centrifugation. The final supernatants from each of the three centrifugations were combined and solid ammonium sulfate was added to the point of 20% saturation. The resulting solution thus derived from the insoluble fraction was subjected to purification on phenyl Sepharose and Q-Sepharose as described above for the soluble fraction. The purified soluble and insoluble fractions were combined to produce about 1.8 mg of purified stromelysin 81-256 fragment (SCD) per gram of original cell paste.

B. Preparation of Yeast Elongation Factor 3 (YEF3) Sub-Fragments Fused with the Green Fluorescent Protein of Aequoria victoria

The carboxy-terminal truncation fragments of EF3 were expressed in E. coli as fusion proteins, with the green fluorescent protein of Aequorea victoria (GFP) fused to the amino terminus of the EF3 fragment. Expression of the desired protein fragment was carried out by growing the transformed E. coli containing the plasmid of interest at 30° C. in enriched media supplemented with the appropriate antibiotics to mid-log phase (OD₆₀₀ 0.4-1.0). Isopropyl-beta-thiogalactopyranoside (IPTG) was added to 1 mM and the cultures were incubated an additional 2-4 hours at 30° C. Cells were harvested by centrifugation and either stored at −80° C. or used immediately for protein isolations.

The fusion proteins were isolated by affinity chromatography on Ni-NTA resin as follows: 100 mL cultures expressing the desired protein were lysed in 10 mL of 50 mM Tris pH 7.8, 500mM NaCl, (lysis buffer) with 1 mM PMSF by either two passes through a french press or treatment for 30 min with 0.1 mg/mL lysozyme followed by 2×2 min sonification cycles using a semi-micro tip (50% duty cycle, power setting 5). The 10,000×g supernatant (soluble fraction) was incubated with 1 mL of resin for ≧1 hour at 4° C. The resin was then poured into a column and washed with 10 mL of lysis buffer. The resin was then washed with 10 mL of 20 mM K₂P0₄, pH 6.0, 500 mM NaCl followed by 10 mL of 20 mM K₂P0₄, pH 6.0, 50 mM imidazole. The protein was eluted with of 20 mM K₂P0₄, pH 6.0, 500 mM imidazole. Fractions containing green color were pooled and immediately diluted with assay buffer (see below). Purity was established using SDS gel electrophoresis.

The GFP-EF3 fusion proteins were dialyzed into 50 mM TrisHCl, 30 mM KCl, 10 mM magnesium acetate, 5 mM 2-mercaptoethanol, 1 mM NaN₃, 10% glycerol, pH adjusted to 7.8 (assay buffer). Ribosomes were isolated from Saccharomyces cerevisiae and resuspended in the same assay buffer. One hundred microliters total solution containing on average 80 nM GFP-EF3 fusion protein and 300 nM ribosomes were then mixed together in a 7 mm×20 mm thick-walled centrifuge tube. Ligand(s) was/were added and the tube contents mixed by vortexing, and then placed in the ultracentrifuge rotor. Following centrifugation under the conditions described above under the heading “General Methodology,” the tube contents were fractionated and analyzed by fluorescence spectrometry.

The steps employed in the preparation of the transformed E. coli for expression of the GFP-EF3 fusion proteins is as follows.

### 1. Plasmid pYEU-EF3 (SEQ. ID No. 6) (Insert is from nt 5621 to nt 8755 Relative to the Linearized Plasmid Sequence Given in SEQ ID No. 6; the Corresponding Insert Protein is SEQ ID No. 7)

A synthetic double stranded oligodeoxyribonucleotide encoding the N-terminal eleven residues of the S. cerevisiae yeast elongation factor 3 protein (EF3), through the natural XbaI site with flanking BamHI and SalI sites, was cloned into the plasmid pYEUra3™ (Clontech, 1020 East Meadow Circle, Palo Alto, Calif. 94303, USA; Genbank accession U02457) as a BamHI/SalI fragment. The resulting plasmid was linearized with XbaI and ligated to a 3.3 kb XbaI fragment derived from the YEF3 gene (Genbank Accession No. 005583). The resulting plasmid, pYEU-EF3 contained the entire coding region of EF3 flanked by BamHI sites and the plasmid SalI and XhoI sites.

### 2. Plasmid pBDEF3 (SEQ ID NO. 8) (Insert is From nt 936 to nt 4070 Relative to the Linearized Plasmid Sequence Given in SEQ ID No. 8; the Corresponding Insert Protein is SEQ ID No. 9)

The 3503 base pair BamHI/XhoI fragment containing the entire EF3 coding sequence was isolated from pYEU-EF3. The BamHI and Xhol ends were ligated to synthetic adapters (N/B and X/R, respectively) to convert the BamHI and XhoI sites to NcoI and EcoRI respectively. The product was ligated into plasmid pAS2-I (Clontech, Genbank accession U30497) linearized with NcoI and EcoRI. The resulting plasmid (pBDEF3) contained the entire EF3 coding sequence fused to the C-terminus of the Gal4 binding domain with a 10 amino acid spacer derived from the pYEU-EF3 adapter and the N/B adapter.

### 3. Plasmid pET28-GFP (SEQ ID No. 10; Insert is From nt 42 to nt 821 Relative to the Linearized Plasmid Sequence Given in SEQ ID No.10; Insert Protein Sequence is SEQ ID No.11)

The coding sequence for green fluorescent protein (GFP) of Aequoria victoria was amplified from the plasmid pEGFP-NI (Clontech, Genbank Accession No. U55762) using PCR with primers Nde-GFP-5′-S (5′GGAATTCCATATGGTGAGCAAGGGCGAGGAGC) (SEQ ID No. 12) and Bsa-GFP-3′-AS (5′-GACTCGGTCTCACTTGTACAGCTCGTCCATGCCG) (SEQ ID No. 13). The sense primer (Nde-GFPS) contains the natural initiation codon for GFP (bold) placed in an Ndel site (underlined, see Table 2). The anti-sense primer (Bsa-GFP-AS) contains the natural stop codon for GFP and a BsaI Class 2S restriction site that will cut the DNA 5′ to the stop codon. The 5′ CTTG overhang generated by Bsal was used to create the fusion.

PCR was performed with Pfu thermostable polymerase (Stratagene, 11011 North Torrey Pines Road, La Jolla, Calif., 92037, USA) in 100 μL of supplied buffer, 0.5 μM each primer, 5 ng template and 0.2 mM each dNTP in a 100 ul reaction for 25 cycles of 95° C., 30s; 55° C., 30s; and 68° C. 30s. The PCR product was purified using the Wizard™ PCR purification system (Promega) according to the manufacturer's recommendations. The purified product was digested with NdeI and ligated to pET28A(+) (Novagen), previously linearized with BamHI, made blunt-ended with the Klenow fragment of E. coli DNA polymerase, and subsequently digested with NdeI. The ligated plasmid was used to transform HMSI 74(DE3) (Novagen) which served both as the source of plasmid for subsequent GFP-EF3 fusions constructs and for expression of the GFP control protein.

### 7. pET-GFP-EF3:980-* Fusions

The DNA coding for the EF3 region of interest along with indicated mutations was amplified using PCR with the primer Bsa-5′-980-S (5′-ACTCGGTCTCACAAGAGTGGTCA AGGTGCTGGTCCAAG, SEQ ID No. 14) and a clone specific primer as indicated in Table 1. The clone specific primers were antisense primers containing a stop codon at the desired location and any additional mutation or sequence required in addition to a restriction endonuclease recognition sequence for directional cloning.

For all constructs, PCR was performed with Pfu thermostable polymerase (Stratagene) with supplied buffer, 0.5 uM each primer, 5 ng template and 0.2 mM each dNTP in a 100 ul reaction for 25 cycles of 95° C., 30s; 55° C., 30s; and 68° C. 30s. PCR products were purified with either QiAquick™ PCR purification kits (Qiagen) or with Wizard™ PCR purification system (Promega) according to the manufacturer's recommendations.

a. pETGFP-EF3:980-1044 (SEQ ID No. 15; Insert is From nt 5268 to nt 6242 Relative to the Linearized Plasmid Sequence Given in SEQ ID No. 15; Insert Protein Sequence is SEQ ID No. 16)

An aliquot of the purified PCR product was digested with restriction endonuclease Bsal (New England Biolabs, Beverly, Mass., USA) followed by BamHI. The double digested PCR products were mixed with Ndel/Bsal digested PCR product containing the GFP sequence amplified with Nde-GFP-S and Bsa-GFP-AS (see above). The mixture was ligated to pET28A linearized with NdeI and BainHI using T4 DNA ligase.

The remaining pETGFP-EF3:980-*, the PCR products obtained from Bsa-5′-980-S and the specific antisense primer were digested with Bsal and the enzyme specific for the anti-sense primer. The double digested PCR products were ligated to pET-GFP, linearized with Bsal and the same clone specific enzyme indicated in Table 2.

Each ligation product was used to transform HMS I 74(DE3) (Novagen) to kanamycin resistance. Transformed cells were used both as a source of plasmid DNA and for expression of the desired fusion protein. All constructs were sequence confirmed using either Sequenase 2 (Amersham Pharmacia Biotech, Inc., 800 Centennial Avenue, P. O. Box 1327, Piscataway, N.J. 08855, USA) or by automated sequencing on a Model 377 Sequencer (Perkin-Elmer Applied Biosystems, 850 Lincoln Centre Drive, Foster City, Calif. 94404, USA).

b. pETGFP-EF3:980-1031 (SEQ ID No. 28; Insert is From nt 5268 to nt 6242 Relative to the Linearized Plasmid Sequence Given in SEQ ID No. 28; Insert Protein Sequence is SEQ ID No. 29)

c. pETGFP-EF3:980-1025 (SEQ ID No. 30; Insert is From nt 5234 to nt 6169 Relative to the Linearized Plasmid Sequence Given in SEQ ID No. 30; Insert Protein Sequence is SEQ ID No. 31)

d. pETGFP-EF3:980-1019 (SEQ ID No. 32; Insert is From nt 5228 to nt 6127 Relative to the Linearized Plasmid Sequence Given in SEQ ID No. 32; Insert Protein Sequence is SEQ ID No.33)

e. pETGFP-EF3:980-1013 (SEQ ID No. 34; Insert is From nt 5234 to nt 6115 Relative to the Linearized Plasmid Sequence Given in SEQ ID No. 34; Insert Protein Sequence is SEQ ID No. 35)

f. pETGFP-EF3:980-1-008 (SEQ ID No. 36; Insert is From nt 5234 to nt 6100 Relative to the Linearized Plasmid Sequence Given in SEQ ID No.36; Insert Protein Sequence is SEQ ID No. 37)

g. pETGFP-EF3:980-1008: 1025-1031 (SEQ ID No. 38; Insert is From nt 5234 to nt 6100 Plus nt 6170 to 6242 Relative to the Linearized Plasmid Sequence Given in SEQ ID No.38; Insert Protein Sequence is SEQ ID No. 39)

TABLE 2

Sequence of the Anti-sense Primers Used with Bsa-5′-980-S for
PCR Amplification of the EF3 Sequence Necessary to
Construct the Indicated GFP-EF3 Fusion Protein

| | | | |
|---|---|---|---|
| GFP-EF3 | | Enzyme Sequence | |
| Fusion | Anti-Sense Primer Sequence | | ID No. |

| | | | |
|---|---|---|---|
| pETGFP-EF3 | 5′-catgggatccTTAGAATTCTTCGTCAGAAG | BAMHI | 17 |
| 980-1044 | | | |
| pETGFP-EF3 | 5′- | XhoI | 18 |
| 980-1031 | catgctcgagttaCTTCTTCTTCTTCATTCTTTC | | |
| | C | | |
| pETGFP-EF3 | 5′- | XhoI | 19 |
| 980-1025 | catgctcgagttaTTCCTTCTTCTTCTTTCTCAA | | |
| | TTCCGC | | |
| pETGFP-EF3 | 5′- | XhoI | 20 |
| 980-1019 | catgctcgagttaCAATTCCGCAGAAGACAAC | | |
| pETGFP-EF3 | 5′- | XhoI | 21 |
| 980-1013 | cargctcgagttaCTTCTTCTTCTTCTTACCACC | | |
| pETGFP-EF3 | 5′-catgctcgagttaACCACCGGCAATCTTGT | XhoI | 22 |
| 980-1008 | | | |
| pETGFP-EF3 | 5′-ggacttaagcttaCTTCTTCTTCTTCATT | HindIII | 23 |
| 980-1031:E125Q | CTTTgCTTCTTCTTCTTTCTC | | |
| pETGFP-EF3 | 5′- | HindIII | 24 |
| 980-1031:E125K | ggacttaagcttaCTTCTTCTTCTTCATTCTTTtC | | |
| | T TCTTCTTCTTTCTC | | |
| pETGFP-EF3 | 5′- | HindIII | 25 |
| 980- | ggacttaagcttaCTTCTTCTTCTTagaTCTTTCC | | |
| 1031:M127S | TTCTTCTTCTTTCTC | | |
| pETGFP-EF3 | 5′-ggacttaagcttaggaagaggaagacatTCTTTCC | HindIII | 26 |
| 980-1031:K128- | TTCTTCTTCTTTCTC | | |
| 31S | | | |
| pETGFP-EF3 | 5′-ggacttaagcttactttttcttcttcatTCTTTCACCAC | HindIII | 27 |
| 980-1008:1025- | CGGCAATCTTGTTAC | | |
| 1031 | | | |

The restriction endonuclease recognition sequence designed into each primer is underlined, the (anti-sense) stop codon and any intended mutation is in bold. Sequence annealing to the native EF3 sequence is in capitals.
The SEQ ID No.’s of the resulting plasmids, their insertion coding regions, and the insertion protein sequences in Table 3.

The SEQ ID No.'s of the resulting plasmids, their insertion coding regions, and the insertion protein sequences are given in Table 3.

TABLE 3

| | SEQ ID | | Insertion Protein |
|---|---|---|---|
| Plasmid | No. | Coding Region* | SEQ ID No. |
| pETGFP-EF3 | 15 | nt 5268 to nt 6242 | 16 |
| 980-1044 | | | |
| pETGFP-EF3 | 28 | nt 5234 to nt 6169 | 29 |
| 980-1031 | | | |
| pETGFP-EF3 | 30 | nt 5234 to nt 6151 | 31 |
| 980-1025 | | | |
| pETGFP-EF3 | 32 | nt 5228 to nt 6127 | 33 |
| 980-1019 | | | |
| pETGFP-EF3 | 34 | nt 5234 to nt 6115 | 35 |
| 980-1013 | | | |
| pETGFP-EF3 | 36 | nt 5234 to nt 6100 | 37 |
| 980-1008 | | | |
| pETGFP-EF3 | 38 | nt 5234 to nt 6100 plus nt | 39 |
| 980-1008: | | 6170 to nt 6242 | |
| 1025-1031 | | | |

*Relative to the linearized plasmid having the given SEQ ID No.

D. Preparation of Erythromycin Resistant Methylase (Erm-AM)

Erm-AM was cloned from a clinical strain of Streptococcus pneumoniae as described by Yu, et al., Natural Structural Biology, 4(6): 483-489 (1997). The gene was sub-cloned into pET-24(+) plasmid (Invitrogen Corporation, 1600 Faraday Ave, Carlsbad Calif., USA 92008) and expressed in E. coli BL21 (DE3)/pV4 cells. Fermentations with E. coli BL21(DE3)/pV4 were carried out at a 10L scale in a Micros Fermentor (Micros, New Brunswick Scientific, Edison, N.J., USA). The growth media consisted of Superbroth supplemented with 2.2 g/L glucose, 10 mg/L thiamine, 1.4 mg/L FeSO₄.7H₂O, 30 mg/L kanamycin, and 0.33 mL.L trace element solution. The trace element solution consisted of (per each liter of 5N HCl) 10 g manganese sulfate monohydrate, 10 g of aluminum sulfate monohydrate, 4 g of cobalt chloride, 2 g of zinc sulfate heptahydrate, 2 g of sodium molybdate dihydrate, 1 g of cupric chloride dihydrate, and 0.5 g of boric acid.

During the fermentation, air was fed to the fermentor at a constant rate of 1 vvm. The dissolved oxygen concentration was maintained at 45% saturation through a cascade control loop which increased the fermentor agitation speed when the dissolved oxygen concentration dropped below 45% saturation. Culture pH was not controlled during the initial growth phase. However, during the expression phase, the pH was controlled at pH 6.70 by the automatic addition of 4N KOH and 4N H2SO₄. When the glucose concentration in the fermentor dropped to 0.5 g/L, automatic feeding of a 20% aqueous glucose solution was initiated. The glucose feed rate was periodically adjusted to ensure that the glucose concentration in the fermentor was within the concentration range of 0.5-1.5 g/L. When the culture A₆₀₀ reached a value of 4.5, expression of ErmAM was induced by the addition of 1 mM IPTG. During the initial growth phase and the first 1 hour and forty minutes of the expression phase, the temperature was maintained at 37° C. After that time, the temperature was controlled at 30° C. Cells were harvested from the fermentor 4 hours after induction. Analysis of the soluble and insoluble fractions of the final cell lysate revealed that approximately 60% of the ErmAM resided in the soluble phase.

The harvested cells were resuspended in 50 mM Tris-HCl buffer, pH 7.8, containing 5 mM DTT, 0.1% sodium azide, 1 mM magnesium chloride, 10% glycerol, 1 mM PMSF, and 20 units/ml of Benzonase® enzyme (Benzon Pharma A/S, Langebjerg 1, DK-4000, Roskilde, DK). The resulting mixture was gently stirred for one hour and then cooled to 4° C. The cells were then sonically disrupted using a 50% duty cycle. After cell lysis EDTA is added to 5 mM. The resulting lysate was centrifuged at 14,000 rpm for 30 minutes . The conductivity of the resultant clarified cell lysate should be around 3-4 millimho. The pH of the lysate was adjusted to pH 8.0 with concentrated sodium hydroxide. The sample is loaded to a S-Sepharose FF column previously equilibrated with starting buffer comprised of 50 mM Tris-HCl buffer, pH 7.8, containing 5 mM DTT, 0.1% sodium azide, and 10% glycerol. The protein is eluted with a linear gradient comprising the starting buffer and 500 mM sodium chloride. The purified protein is then concentrated and stored at −4° C. for subsequent use.

IV. Illustrative Examples of the Method of the Invention

### A. EXAMPLE 1

### Determination of Affinity Binding to the Catalytic Domain of Stromelysin (SCD) of Individual Compounds

The affinity of each of four individual compounds for the catalytic domain of stromelysin was determined by the spin-screen method of this invention, using HPLC as the method of determining the concentrations following centrifugation.

Stromelysin catalytic domain for these experiments was isolated and refolded from inclusion bodies produced recombinantly in E. coli as described above. The protein was dialyzed into 20 mM NH₄HCO₃, 10 mM CaCl₂, 5 mM 2-mercaptoethanol, pH adjusted to 7.2 with acetic acid. The SCD and ligands were mixed in a 7 mm×200 mm thick-walled centrifuge tube, and sucrose was added to a concentration of 0.5% to help stabilize the protein gradient after centrifugation.

One-hundred microliters of 100 μM protein was placed in individual centrifuge tubes in 1 μL amounts from a dimethyl sulfoxide stock solution until the concentration of ligand was equal to that of the protein (100 μM). The tube contents were mixed by vortexing and then placed in a TLA-100 rotor, centrifuged, and the concentrations of the compounds at each of five depths of the centrifuge tube measured by HPLC. The centrifugation and HPLC analyses were carried out according to the methods detailed under the heading “General Methodology” described above. The identity of each compound, and their affinities, previously determined by another method, appear in Table 4.

TABLE 4

| | | Affinity |
|---|---|---|
| FIG. | Compound | (Kᵢ) |
| 2A | 3-[4-cyano(1,1-biphenyl-4-yl)oxy]-N- | ˜25 nM |
| | hydroxypropanamide | |
| 2B | 5-[4-cyano(1,1-biphenyl-4-yl)oxy]-N- | ˜3.5 μM |
| | hydroxypentanamide | |
| 2C | 4′-hydroxy-1,1-biphenyl-4-carbonitrile | ˜1 mM |
| 2D | N-phenylbenzamide | Non- |
| | | binding |

Each compound was first spun with buffer only, and the concentration gradient of the compound determined by HPLC. Next, each compound was spun in buffer together with SCD. The results of these experiments appear in FIGS. 2A-2D, with the data for the compound spun in the absence of protein shown as open circles, and the data for compound in the presence of SCD shown as closed squares.

### B. EXAMPLE 2

### Determination of Affinity Binding to Erythromycin Resistant Methylase (Erm AM) of Both Soluble and Insoluble Ligands

I. Soluble Ligands

In this experiment, the interaction of compounds known from the prior art to bind to Erm AM and soluble in the centrifuge carrier solvent were detected. The analysis was carried out with a 25-compound library using the spin-screen method of the present invention with the compound concentration gradients being determined by mass spectrometry. Sample preparation, centrifugation, analysis by mass spectrometry, and data analysis were according to the techniques described above under the heading “General Methodology.” The results are shown in FIGS. 3A and 3B. The figures represent planar cuts of the three-dimensional surface contour plot defined by the three axes: m/z, sample fraction, and mass spectral signal intensity. The planar cuts are taken at comparatively high values constant values of m/z. That is to say, at m/z signal levels which detected only the most strongly binding ligands in the 25-compound library.

FIGS. 4A and 4B show concentration gradients for N-cyclohexyl-6-(1-piperidinyl)-1,3,5-triazine-2,4-diamine (m/z=277) and 4-[4-amino-6-(clyclohexylamino)-1,3,5-triazin-2-yl]phenol (m/z=286).

1. Insoluble Ligands

In this experiment, the interaction of compounds known from the prior art to bind to Erm AM, but insoluble in the centrifuge carrier solvent were analyzed. The analysis was carried out with a 25-compound library using the spin-screen method of the present invention with the compound concentration gradients being determined by mass spectrometry. Sample preparation, centrifugation, analysis by mass spectrometry, and data analysis were according to the techniques described above under the heading “General Methodology.” The results are presented in FIGS. 5A and 5B.

### C. EXAMPLE 3

### Determination of Affinity Binding to Erm AM by Analysis of Mixtures of Compounds

In this experiment, a 25-compound library of compounds was tested for binding to Erm AM by the spin-screen method of the present invention. The capability of the compounds to bind to the protein in this experiment was not known prior to the commencement of the experiment. Sample preparation, centrifugation, and compound detection by mass spectrometry were as described in the section titles “General Methodology” above. The four compounds found by this experiment to bind to Erm AM, but not previously known to possess that property, were 4-amino-6-chloro-1,3-benzenesulfonamide (m/z=312, 314); 4-[[[(3-trifluoromethyl)-phenyl]amino]carbonyl]amino]benzoic acid (m/z=324); 4-hydroxy-1-[4-(phenylmethoxy)-phenyl]-2(1H)-quinoline (m/z=347), and 1,2-benzenedicarboxylic acid, mono [(3-chloro-phenyl)phenylmethyl] ester (m/z=366).

The data from the experiments are shown at a selected value of m/z in the 3-(dimensional plot of the mass spectral data array shown in FIGS. 6A and 6B.

### D. EXAMPLE 4

### Determination of Information About the Specific Region of Yeast Elongation Factor 3 (EF3) Required for Binding to Yeast Ribosomes

The GFP-EF3 fusion proteins prepared as detailed above under the heading “Preparation of Starting Materials” were dialyzed into 50 mM Tris.HCl, 30 mM KCl, 10 mM magnesium acetate, 10 mM 2-mercaptoethanol 1 mM sodium azide, 10% glycerol with pH adjusted to 7.8 (assay buffer). Ribosomes were isolated from Saccaromyces cervisae and resuspended in the same buffer. One hundred microliters total solution containing on average 80 nM GFP-EF3 fusion protein and 300 nM ribosomes were then mixed together in a 7 mm×20 mm thick-walled centrifuge tube. Ligands were then added as needed. The tubecontents were then mixed by vortexing and placed into a TLA-100 rotor.

The mixture was subjected to centrifugation at a speed and time required to clear the solution of the ribosomes but not the GFP-EF3 fusion protein, in this case, 20 minutes at 70,000 rpm. The speed and time necessary for ribosome clearance was estimated from the clearing factor of the rotor (k) and sedimentation coefficient of the ribosome (S_{20,w}), S_{20,w} was taken to be 80S: The tubes were then carefully removed and placed vertically in a rack.

Four fractions of 20 uL each were removed by hand and dispensed into microfuge tubes. Twenty microliters of assay buffer was added to the final (fifth) 20 uL fraction and the tube vortexed to resuspend the ribosome pellet.

Fluorescence of the GFP-EF3 fusion protein in each of the five fractions from the samples was examined using a SLM-Abt-2 spectrofluorimeter. Emission was monitored at 512 nm (4 nm slit width), with excitation of 488 nm (4 nm slit width), as each of the five fractions was sequentially examined. For the first four fractions, 10 uL was diluted to a final volume of 300 uL prior to examination. For the fifth fraction, 20 uL was diluted to a final volume of 300 uL.

The plots of the fluorescent spectroscopic analyses appear in FIG. 7.

While there have been shown and described what are believed to be the preferred embodiments of the method of this invention, it will be apparent to those of ordinary skill in the art to which this specification is directed, that various modifications can be made without departing from the scope of the invention as it is encompassed by the appended claims.

We claim:

1. A method of determining affinity binding comprising the steps of:
a) exposing at least one target substance to at least one putative ligand substance in a mixture in a container vessel;
b) subjecting the container vessel and contents to centrifugation; and
c) subsequently determining a physical parameter at each of several depths of the mixture in the container vessel which is proportional to the concentration of the at least one target substance or the at least one putative ligand substance in the mixture.

2. The method according to claim 1 wherein the target substance is selected from the group consisting of biopolymers, viruses, cells, and cell components.

3. The method according to claim 2 wherein the target substance is a biomolecule selected from the group consisting of lipids, lipoproteins, polysaccharides, proteins, DNA, and RNA.

4. The method according to claim 2 wherein the target substance comprises cells or sub-cellular components.

5. The method of claim 4 wherein the target substance comprises cell walls or cell wall components.

6. The method of claim 4 wherein the target substance comprises a cell organelle.

7. The method of claim 6 wherein the cell organelle comprises ribosomes.

8. The method of claim 1 wherein the at least one putative ligand substance is a compound having a molecular weight less than about 1 kDa.

9. The method according to claim 3 wherein the at least one putative ligand is selected from the group consisting of polysaccharides, proteins, DNA, and RNA.

10. The method according to claim 4 wherein the at least one putative ligand is selected from the group consisting of polysaccharides, proteins, DNA, and RNA.

11. The method according to claim 5 wherein the at least one putative ligand is selected from the group consisting of polysaccharides, proteins, DNA, and RNA.

12. The method according to claim 4 wherein the at least one putative ligand compound is a virus.

13. The method of claim 1 wherein the step of subjecting the container vessel and contents to centrifugation is carried out at a rotational rate and for a time sufficient to clear the solution of the at least one target substance, but insufficient to clear the solution of the at least one putative ligand.

14. The method of claim 1 wherein the step of subsequently determining a physical parameter at each of several depths of the mixture of the at least one target substance or the at least one putative ligand substance comprises analysis of aliquot samples withdrawn from various depths of the mixture in the container vessel.

15. The method of claim 1 further comprising the step of separating the contents of the container vessel into discrete fractions based upon the depth of the fraction in the mixture in the container vessel prior to the step of determining a physical parameter which is proportional to the concentration of the at least one target substance or the at least one putative ligand substance the fraction.

16. The method of claim 1 wherein the step of determining a physical parameter at each of several depths of the mixture in the container vessel which is proportional to the concentration of the at least one target substance or the at least one putative ligand substance in the mixture is an analytical technique selected from the group consisting of mass spectrometry, nuclear magnetic resonance spectrometry, absorbance spectroscopy, fluorescence spectroscopy, atomic absorption spectroscopy, chromatography, gel electrophoresis, enzymatic assay, immunoassay, and radio-isotopic assay.

17. The method of claim 16 wherein the step of determining a physical parameter at each of several depths of the mixture in the container vessel which is proportional to the concentration of the at least one target substance or the at least one putative ligand substance in the mixture is mass spectrometry.

18. The method of claim 16 wherein the step of determining a physical parameter at each of several depths of the mixture in the container vessel which is proportional to the concentration of the at least one target substance or the at least one putative ligand substance in the mixture is chromatography.

19. The method of claim 16 wherein the step of determining a physical parameter at each of several depths of the mixture in the container vessel which is proportional to the concentration of the at least one target substance or the at least one putative ligand substance in the mixture is absorbance spectroscopy.

20. The method of claim 16 wherein the step of determining a physical parameter at each of several depths of the mixture in the container vessel which is proportional to the concentration of the at least one target substance or the at least one putative ligand substance in the mixture is fluorescence spectroscopy.

21. The method of claim 16 wherein the step of determining a physical parameter at each of several depths of the mixture in the container vessel which is proportional to the concentration of the at least one target substance or the at least one putative ligand substance in the mixture is gel electrophoresis.

22. The method of claim 16 wherein the step of determining a physical parameter at each of several depths of the mixture in the container vessel which is proportional to the concentration of the at least one target substance or the at least one putative ligand substance in the mixture is immunoassay.

23. The method of claim 16 wherein the step of determining a physical parameter at each of several depths of the mixture in the container vessel which is proportional to the concentration of the at least one target substance or the at least one putative ligand substance in the mixture is radio-isotopic assay.

24. The method of claim 18 wherein the step of determining a physical parameter at each of several depths of the mixture in the container vessel which is proportional to the concentration of the at least one target substance or the at least one putative ligand substance in the mixture is high performance liquid chromatography.

25. A method of selecting one or more compounds which demonstrate affinity binding to a pre-selected target substance comprising the steps of:
a) exposing at least one target substance to a mixture of compounds in a a container vessel;
b) subjecting the container vessel and contents to centrifugation;
c) collecting a data array of full scale mass spectral scans of signal intensities for each of several samples collected from the contents of the container vessel at successively greater depths of the container vessel;
d) displaying the data array as a 3-dimensional plot in which one axis represents m/z, the second axis represents depth in the container vessel, and the third axis represents mass spectral signal intensity; and
e) viewing the 3-dimensional plot at one or more planar cuts taken at one or more constant values of m/z in the 3-dimensional plot to select one or more compounds

26. The method of claim 25 further comprising the step of analyzing the data array collected in step c) by a program which determines relative intensities of mass spectral signals from samples collected from the top of the container vessel and samples collected from the bottom of the container vessel and tabulates those compounds for which the relative intensities exceeds a predetermined value.

27. The method according to claim 25 wherein the target substance is selected from the group consisting of biopolymers, viruses, cells, and sub-cellular components.

28. The method according to claim 27 wherein the target substance is a biopolymer selected from the group consisting of lipids, lipoproteins, polysaccharides, proteins, DNA, and RNA.

29. The method according to claim 27 wherein the target substance comprises cells or sub-cellular components.

30. The method according to claim 27 wherein the target substance comprises cell walls or cell wall components.

31. The method according to claim 27 wherein the target substance comprises a cell organelle.

32. The method according to claim 31 wherein the cell organelle comprises ribosomes.

33. A method of determining, in a protein known to bind to a pre-selected target substance, a specific region of the protein required for binding to the target substance, the method comprising the steps of:
a) preparing a fragment of the protein tagged at one terminus with a marker, and having a pre-determined number of aminoacyl residues deleted from the opposite terminus;
b) exposing the tagged protein fragment to the pre-selected target substance in a a container vessel;
c) subjecting the container vessel and contents to centrifugation;
d) determining the concentration gradient of tagged protein in the container vessel by an analytical technique sensitive to the detectable marker;
e) iterating steps a) through d) on successively shorter fragments of the protein until the concentration gradient data indicate the absence of binding between the protein fragment and the pre-selected target substance.

34. The method of claim 33 wherein said pre-determined target substance is selected from the group consisting of a biopolymer, cells, and sub-cellular components.

35. The method of claim 33 wherein the pre-determined target substance is a biopolymer selected from the group consisting of proteins, DNA and RNA.

36. The method of claim 33 wherein the pre-determined target substance is a sub-cellular component.

37. The method of claim 34 wherein the pre-determined target substance comprises cell walls or cell wall components.

38. The method of claim 34 wherein the pre-determined target substance comprises ribosomes.

39. The method of claim 33 wherein the marker comprises radioisotopic labeling.

40. The method of claim 33 wherein said marker comprises an absorbent chromophore.

41. The method of claim 33 wherein said marker comprises a fluorescent chromophore.

42. The method of claim 34 wherein the fluorescent chromophore comprises a fluorescent protein or protein fragment fused with the protein fragment being analyzed for binding to the pre-determined target substance.

43. The method of claim 42 wherein said fluorescent protein or protein fragment is green fluorescent protein (GFP) of Aequoria victoria.
